(19) United States
(12) Patent Application Publication
(10) Pub. No.: US 2018/0015203 A1
(43) Pub. Date: Jan. 18, 2018

(54) SELF-ASSEMBLED ORGANOSILANE COATINGS FOR RESORBABLE METAL MEDICAL DEVICES

(71) Applicant: UNIVERSITY OF PITTSBURGH-OF THE COMMONWEALTH SYSTEM OF HIGHER EDUCATION, PITTSBURGH, PA (US)

(72) Inventors: ELIA BENIASH, MT. LEBANON, PA (US); AVINASH JAGANNATH PATIL, PITTSBURGH, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 15/548,146

(22) PCT Filed: Feb. 3, 2016
(86) PCT No.: PCT/US2016/016292

§ 371 Date: Aug. 2, 2017

Related U.S. Application Data
(60) Provisional application No. 62/111,165, filed on Feb. 3, 2015.

(51) Int. Cl. A61L 27/34; A61L 27/54; A61L 27/04; A61L 27/58

(52) U.S. Cl. CPC A61L 27/34; A61L 27/58; A61L 27/54; A61L 27/047; A61L 2400/18; A61L 2420/02

(57) ABSTRACT

The invention relates to self-assembled organosilane coatings for resorbable medical implant devices. The coatings can be prepared from coating compositions containing organosilane and can be applied to metal or metal alloy substrates. Prior to applying the coatings, the surfaces of the substrates can be pre-treated. The coatings can be functionalized with a binding compound that is coupled with an active component. The coatings can be applied using various techniques and apparatus, more particularly, by a deep-coating process conducted at ambient conditions.

### CROSS-REFERENCE TO RELATED APPLICATIONS

This application claims priority under 35 U.S.C. §119(e) to U.S. Provisional Patent Application Ser. No. 62/111,165, filed Feb. 3, 2015, entitled “Self-Assembled Organosilane Coatings for Resorbable Metal Medical Devices”, which is herein incorporated by reference.

### GOVERNMENT SUPPORT

This invention was made with government support under Grant No. EEC0812348 awarded by the National Science Foundation (NSF). The government has certain rights in the invention.

### FIELD OF THE INVENTION

The present invention relates to self-assembled organosilane-containing compositions, methods of preparing the compositions, methods of depositing/applying the compositions on a substrate to form a coating, and uses for the coated substrates as medical implant devices.

### BACKGROUND OF THE INVENTION

Every year millions of orthopedic and craniofacial surgical procedures are performed in the United States, which require placement of metal, e.g., stainless steel or titanium, hardware in a patient body. After bone healing is complete, these metal implant devices are no longer needed. The devices can be left in situ or, alternatively, they can be removed. Each of these alternatives has disadvantages or problems associated therewith. For example, leaving the hardware in situ increases the chances of infection and rejection, and removal of the hardware requires a second surgery and causes a risk of infection, pain and discomfort to the patient, as well as it being an additional expense. To overcome these disadvantages or problems, there has been developed a number of resorbable polymeric devices that are effective to degrade over a period of time. Thus, the device does not remain in-situ and there is no need to surgically removing the device because when the device is no longer needed, the polymeric material degrades or dissolves within the patient body. However, there are also disadvantages associated with the resorbable polymer devices. For instance, it has been found that the resorbable polymeric materials, which are used for die construction of biodegradable medical implant devices, can lack mechanical strength as compared to that exhibited by metal implants and have a limited set of applications. As a result, there is an interest in the art to identify materials that degrade over time while demonstrating sufficient mechanical strength prior to degradation.

It has been found that the development of new technologies for implantable devices based on resorbable magnesium and magnesium alloys has the potential to make a significant, clinical impact Magnesium and magnesium alloys are suitable materials for the construction of resorbable devices because they have mechanical properties compatible to bone and can be resorbed over a period of time. However, there are other properties of magnesium and magnesium alloys that are problematic for their use as medical implant devices. For example, magnesium is not typically used in the fabrication of medical implant devices primarily because the corrosion of magnesium results in the production of hydrogen. Medical implant devices constructed of magnesium can cause the accumulation of hydrogen in areas surrounding the device and thus, result in the formation of gas cavities in the patient body. In order for magnesium and magnesium alloys to be considered as suitable materials for use in constructing medical implant devices, the rate of corrosion of these materials needs to be closely monitored and controlled to prevent formation of gas cavities. Thus, there are a number of important characteristics that have to be controlled in order to achieve the best clinical outcomes including, for example, rate of resorption, control of corrosion products, tissue integration and osteoconduction properties of the device.

It is known to deposit a coating composition on the surface of metal implant devices to modify the properties, e.g., corrosion, of the devices. Coatings for metal-based implants have been classified as conversion or deposition coatings. Conversion coatings are generally formed in situ through a reaction between the substrate and its environment, and are typically inorganic. For application to magnesium or magnesium alloys, these coatings are often composed of oxides, phosphates or fluorides. Conversion coatings typically advantageously exhibit good adhesion so the substrate, however, there are disadvantages associated with mechanical durability and biocompatibility of these coatings. Deposition coatings are typically organic or ceramic and are applied through physical interactions with the surface of a metal substrate. For application to magnesium or magnesium alloy substrates, deposition coatings often require a conversion coating pre-treatment to improve adhesion to the alloy substrates. In the absence of a conversion coating pre-treatment, e.g., one-step coatings, it is likely that the coated substrate will demonstrate poor adhesion and corrosion protection.

There is a desire in the art to develop a mechanism for controlling the rates of corrosion of magnesium and magnesium alloy in order to reduce or minimize the production and accumulation of hydrogen resulting therefrom, and to construct medical implant devices from materials that demonstrate sufficient mechanical strength when needed and degradation over time when no longer needed. Further, there is a desire to develop a coating that is effective to control rates of corrosion of magnesium and magnesium alloy and to reduce or minimize the production and accumulation of hydrogen resulting therefrom, and that demonstrates good adherence or adhesion to the magnesium and magnesium alloy.

### SUMMARY OF THE INVENTION

An object of the present invention is to develop novel coating compositions for application to magnesium and magnesium alloy substrates for use as medical implant devices. In particular, an object of the present invention is to develop hybrid bio-inspired anticorrosive coatings based on self-assembled multilayer organosilane. The surface of these coatings can be modified via covalent bonding with an active component, including bioactive molecules, such as proteins and peptides. These surface chemistry modifications can provide the ability to control different physical chemical properties of the coatings, including but not limited to, hydrophobicity and charge, as well as bioactivity. These coatings can effectively control the degradation rate of magnesium and magnesium alloy resorbable devices to insure safety and efficiency, and to induce desirable tissue responses. Further, these coatings can be functionalized to regulate the rate of corrosion and insure the device integration into target tissues.

In one aspect, the invention provides a medical implant device including a substrate including metal and having an outer surface, a self-assembled organosilane-containing coating applied to the substrate, a binding compound combined with the coating, and an active component coupled to the binding compound.

The metal can he selected from the group consisting of magnesium and magnesium alloy.

The device can further include a pretreatment applied to the outer surface of the substrate and the coating can be applied to the pretreatment. The pretreatment can be selected from the group consisting of polishing with nitric acid, etching with nitric acid, passivating with sodium hydroxide, and combinations thereof.

The coating can include alkyltriethoxysilane. The alkyltriethoxysilane can have a tail including a C₄₋C₂₀ aliphatic backbone and a silane head. The coating can include a co-polymer of decyltriethoxysilane and tetramethoxysilane.

The binding compound can include 3-aminopropyl-trimethoxysilane. In certain embodiments, the binding compound is selected from the group consisting of amine, carboxyl, thiol, hydroxyl and mixtures thereof. The binding compound can be coupled to a surface of the coating or mixed with the composition that forms the coating.

In another aspect, the invention provides a method of preparing a medical implant device. The method includes obtaining a uncoated substrate having an outer surface, preparing a coating composition including organosilane, applying the coating composition to the uncoated substrate to form a coating thereon, functionalizing the coating with a binding compound, and coupling an active component to the binding compound.

The step of applying the coating composition can be conducted by a deep-coating process at ambient temperature.

The steps of preparing and applying the coating composition can include combining the organosilane and solvent to form a solution; applying the solution to the uncoated substrate by dipping or immersing the substrate into a bath of the solution for a time sufficient for the organosilane to bond to the substrate; evaporating the solvent; inducing the organosilane to self-assemble into a micro- or nano-structure; and forming a thin film coated substrate.

The method can further include pretreating the surface of the uncoated substrate prior to applying the coating composition.

### BRIEF DESCRIPTION OF THE DRAWINGS

FIGS. 1A, 1B, 1C and 1D are plots showing cumulative hydrogen release profiles from samples following incubation periods, in accordance with certain embodiments of the invention;

FIGS. 2A and 2B are bar graphs showing percent of weight loss in samples following incubation periods, in accordance with certain embodiments of the invention;

FIG. 3 is a photograph of a mouse at Day 0 and Day 7 showing gas pocket formation, in accordance with certain embodiments of the invention; and

FIG. 4 is a bar graph showing fluorescence intensity of coated, functionalized samples, in accordance with certain embodiments of the invention.

### DETAILED DESCRIPTION OF THE INVENTION

The invention generally relates to medical implant devices, self-assembled organosilane-containing coating compositions, coated medical implant: devices; methods of applying/depositing the coating compositions onto the medical implant devices and, systems and methods of coupling bioactive agents to the surface of the coated medical implant devices. The medical implant devices can be composed of a wide variety of materials that are known in the art for such purposes. In accordance with the objectives of controlling the rates of corrosion of magnesium and magnesium alloy in order to reduce or minimize the production and accumulation of hydrogen resulting therefrom, and to construct medical implant devices from materials that demonstrate sufficient mechanical strength when needed and degradation over time when no longer needed, it is preferred that the medical implant devices be composed of magnesium or magnesium alloy. The coating compositions can be directly applied to, or deposited on, the surface of the medical implant devices, i.e., in the absence of any pretreatment of pre-coating of the surface. However, for the purpose of improving the adherence and or adhesion of the coatings to the surface of the devices, it is preferred to perform pretreatment or pre-coating of the devices' surface.

Without being bound by any particular theory, it is believed that the coatings are effective to modify various properties and characteristics of the underlying magnesium-containing substrate of the devices. For example, the coatings can be effective to control one or more of the following properties of the magnesium-containing substrates: corrosion rate, production/accumulation of hydrogen, calcium phosphate precipitation, rate of resorption, tissue integration and osteoconduction. In certain embodiments, the coatings can be effective to reduce or preclude the corrosion rate and, in turn, the production/accumulation of hydrogen, as well as reducing calcium phosphate formation around the device. Further, the surface of these coatings can be modified via covalent bonding with different molecules, including bioactive molecules, such as proteins and peptides. These surface chemistry modifications can provide the ability to control different physical chemical properties of the coatings,including but not limited to, hydrophobicity and charge, as well as bioactivity. Furthermore, the invention relates to the use of the coated magnesium-containing substrates in constructing/fabricating medical implant devices for use in various surgical applications, such as, but not limited to, dental, orthopedic, craniofacial, and cardiovascular.

Conventional apparatus and techniques are generally known for preparing and applying/depositing a silane coating composition onto a substrate, and for modifying or functionalizing the surface of the formed silane coating. For example, the use of various amphiphilic organosilanes to form nanostructured films for glass coating applications and the application of organosilanes for corrosion control are known. However, there is a need in the art to develop organosilane-containing compositions for use in coating resorbable metallic, e.g., magnesium and magnesium alloy, medical implant devices. In particular, the coatings for medical implant devices require special properties, such as, the ability to adapt to the intrinsically unstable physical and chemical environment of a corroding metal implant device, as well as the ability to be functionalized with bioactive molecules.

In general, self-assembled coatings, e.g., monolayers, are thin films produced by deposition of materials, such as, organosilanes. The coatings are formed, e.g., spontaneously, on surfaces by adsorption and include a head group, tail and functional end groups. The head group can be in a vapor phase or a liquid phase. The head group assembles onto the substrate, while the tail group organizes and assembles farther from the surface of the substrate. The substrate and head group are selected to react with each other. In certain embodiments, a hydrophilic end (e.g., head group) may bond with the substrate surface while a hydrophobic end may be opposite the hydrophilic end.

The self-assembled coating compositions include organosilane, e.g., hybrid organosilanes. In certain embodiments, the coating compositions include amphyphilic organosilane having an aliphatic tail containing a backbone of 4 to 20 carbon atoms (i.e., C₄ to C₂₀) and a silane head. Non-limiting examples of suitable organosilanes include alkylsilanes. In certain embodiments, the coating compositions include alkyltrialkoxysilane, such as, but not limited to, decyltriethoxysilane. The alkylsilane including alkyltrialkoxysilane, e.g., decyltriethoxysilane, can be co-polymerized with another polymer component such as, but not limited to, tetramethoxysilane (TMOS). Further, in certain embodiments, the alkylsilane, e.g., alkyltrialkoxysilane, is combined with a crosslinking material, such as, but not limited to, a UV crosslinking agent.

The self-assembled, coating compositions are applied or deposited onto the magnesium or magnesium alloy surface, e.g., of the medical implant device. The magnesium alloy may be selected from a wide variety of alloys known in the art for constructing medical implant devices. Non limiting examples of suitable magnesium alloys include those magnesium-containing compositions described in PCT Application having International Application No. PCT/US2012/058939 entitled “Biodegradable Metal Alloys” filed on Oct. 5, 2012 and based on U.S. Provisional Patent Application 61/544,127 entitled “Biodegradable Metal Alloys” filed on Oct. 6, 2011, which are incorporated in their entirety herein by reference.

In certain embodiments, the magnesium alloys include elemental magnesium and one or more other elemental components, such as, but not limited to, iron, zirconium, manganese, calcium, yttrium and zinc. The amount of each of the components can vary and, in general, the amounts are selected such that the resulting magnesium alloys are within acceptable non-toxic limits, sufficiently biocompatible and degradable over a period of time.

In general, the self-assembled organosilane coatings can be formed using known apparatus and conventional coating techniques, including, but not limited to, physical vapor deposition, electro-deposition or electro-less deposition. For example, a self-assembled coating can be formed on a magnesium or magnesium alloy substrate at ambient conditions by spinning, dipping or spraying techniques, which are known in the art. In certain embodiments, a coating is formed by employing a deep-coating process at ambient conditions. This process includes combining organosilane and solvent, e.g., water, to form a solution and applying the solution to a magnesium or magnesium alloy substrate by dipping/immersing the substrate in to a hath of die solution. The immersion can be for a time period ranging from minutes to hours and, typically includes sufficient time to allow the organosilane to bond to the substrate. Subsequent evaporation of the solvent, by conventional methods, induces the organosilane to self-assemble into micro- or nano-structures and thin film. The resulting coating, e.g., thin film, is rigid, uniform and has a thickness that can vary from about 100 nanometers to tens of micrometers. The thickness can depend on various factors including the organosilane composition components, the process conditions and the intended use of the coated substrate. In one embodiment, the coating has a thickness of about 1 um. Further, the coating, e.g., laminar structure, can include multiple layers, hi certain embodiments, the coating may be composed of about 30 nm thick layers. Furthermore, the coating can be hydrophobic which may be particularly beneficial for cardiovascular applications.

The coating process in accordance with the invention can optionally include pre-treating or pre-coating the surface of the substrate prior to applying/depositing the organosilane coating composition thereto. The pre-treatment or pre-coating is applied to, or deposited on, the bare, e.g., uncoated, surface of the magnesium or magnesium alloy substrate. The pre-treatment/pre-coating step can vary and may be selected from known pretreatment compounds/compositions, techniques and processes that are employed to improve adherence or adhesion of a coating to the surface of a substrate. In certain embodiments, the pretreatment includes polishing and/or etching the uncoated substrate with nitric acid, and/or passivating with sodium hydroxide. Without, intending to be bound by any particular theory, it is believed that pretreating the substrate prior to applying the coating composition results in a more uniform coating having improved adhesion or adherence properties, as compared to coatings that are formed in the absence of pretreating the substrate.

The coating in accordance with the invention has numerous advantages as compared to conventional coating technology, including, but not limited to, for example, tunability. The thickness of the coating and its mechanical properties can be tuned or controlled. For example, using organosilanes with UV crosslinkable groups provides the ability to increase stiffness simply by exposure to a UV source. Further, copolymerizing organosilanes with tetramethoxysilane produces liquid-like coatings having increased flexibility, which may be particularly useful for cardiovascular applications.

Furthermore, the surface of the coatings can be modified or functionalized to attach or bind an active component to the surface of the coatings. A binding compound, such as, but not limited to amine, carboxyl, thiol, hydroxyl and mixtures thereof, can be used to bind one or more active components to the coatings. In certain embodiments, the binding compound is attached to the surface of the coating. For example, a plurality of molecules containing silane groups, e.g., aminosilanes, such as, but not limited to aminopropyl-trimethoxysilane, can be covalently attached to the surface of the coating to provide chemistry for attachment of the active component, such as, but not limited to alkaline phosphatase, or for modifying hydrophobicity of the surface. In certain other embodiments, the binding compound can be permeated or encapsulated within the composition that forms the coating.

As used herein, the term “active component” and related terms refer to a molecule, compound, complex, adduct and/or composite that exhibits one or more beneficial activities, such as, therapeutic activity, diagnostic activity, biocompatibility, corrosion-resistance, and the like. Active components that exhibit a therapeutic activity can include bioactive agents, pharmaceutically active agents, drags and the like. Non-limiting examples of bioactive agents include, but are not limited to, bone growth promoting agents, such as growth factors, drugs, proteins, antibiotics, antibodies, ligands, DNA, RNA, peptides, enzymes, vitamins, cells and the like, and combinations thereof.

With the binding of one or more active components, the coatings and coated medical implant devices, can be effective to combine anti-corrosion properties with bioactive surface modifications, which can facilitate improved tissue integration and induce desired biological responses.

Organosilane-coated magnesium-containing substrates, in accordance with the invention, are generally effective for tissue regeneration and, in particular, bone regeneration, within a body of a patient. These substrates can be employed as materials of construction for various medical implant devices. Non-limiting examples of suitable medical devices include, but are not limited to, scaffolds, plates, meshes, staples, screws, pins, tacks, rods, suture anchors, tubular mesh, coils, x-ray markers, catheters, endoprostheses, pipes, shields, bolts, clips or plugs, dental implants or devices, such as but not limited to occlusive barrier membranes, graft devices, bone-fracture healing devices, bone replacement devices, join replacement devices, tissue regeneration devices, cardiovascular stents, nerve guides, surgical implants and wires.

It will be appreciated by those skilled in the art that changes could be made to the embodiments described above without departing front the broad inventive concept thereof. It is understood, therefore, that this invention is not limited to the particular embodiments disclosed and the following examples conducted, but it is intended to cover modifications that are within the spirit and scope of the invention.

### EXAMPLES

### Example 1

### Coating Preparation

Hybrid self-assembled organosilane coatings were generally prepared according to known procedures. Amphyphilic organosilane decyltriethoxysilane (DTES) and tetramethoxysilane (TMOS) were co-polymerized over 90 minutes to form a hybrid organosilane solution. Mg alloy disks were obtained. Some of the disks were pre-treated by polishing and etching with nitric acid, and passivating with NaOH prior to applying the solution and forming the coating thereon. Ail of the disks were dip-coated in the silane solution and dried at 37° C. It was found that the surface preparation, e.g., pretreatment, of the disks had a significant effect on the quality of the coatings formed. The disks that were coated without any surface pretreatment exhibited poor coating quality. The coatings had multiple cracks and were easily peeled from the disk surfaces. The disks that included surface pretreatment prior to coating provided coatings that exhibited significant improvements as compared to the disks without pretreatment.

### Example 2

### Assessment of Corrosion Dynamics—H₂ Evolution Method

Disks composed of the following alloys; 99.9 Mg, AZ31, LA2, ZEK100and LA63, were commercially obtained and evaluated. These alloys represented a spectrum of corrosion rates. As described in Example 1, the hybrid organosilane solution was prepared and applied to the disks. It was observed that coating the alloy disks with the hybrid organosilane film significantly reduced the hydrogen evolution as compared to uncoated alloy disks. The reduction was highest in the initial 24 hours, when a burst of H₂ causing the formation of gas pockets was observed. The results clearly indicated the potential of the hybrid organosilane coatings to minimize gas pockets associated with medical implant devices.

### Example 3

### Potentiodynamic Polarization

The effectiveness of hybrid organosilane coatings for corrosion prevention was tested. Disks composed of the following alloys: 99.9% Mg and AZ31, were commercially obtained and evaluated. A control disk was not coated with the hybrid organosilane solution, e.g., a bare disk. Other disks were prepared as follows: (i) not coated with the hybrid organosilane solution, but passivated in NaOH; (ii) coated with the hybrid organosilane solution; (iii) coated with the hybrid organosilane solution and animated; (iv) coated with the hybrid organosilane solution and passivated; (v) coated with the hybrid organosilane solution, animated and passivated. Table 1 shows the results for the AZ31 disks.

TABLE 1
Corrosion Potential (E_{corr}) and Current Density (I_{corr}) Values (for
non-coated and coated Mg substrates).

| Treatment | E_{corr}(V) (SD) | I_{corr} (μA/cm⁻²) (SD) |
|---|---|---|
| Mg bare | −1.77 (0.014) | 171.97 (26.40) |
| Mg—OH | −1.75 (0.027) | 151.84 (31.78) |
| Mg—OH-AS | −1.78 (0.020) | 10.85 (3.93) |
| Mg—OH-AS-APES | −1.80 (0.038) | 3.66 (2.56) |
| AZ31 bare | −1.49 (0.009) | 96.43 (65.90) |
| AZ31-OH | −1.54 (0.032) | 100.84 (50.26) |
| AZ31-OH-AS | −1.62 (0.014) | 6.06 (1.13) |
| AZ31-OH-AS-APES | −1.59 (0.006) | 4.07 (3.04) |

The results in Table 1 demonstrate that the hybrid organosilane coating significantly reduced the corrosion current and this effect was most pronounced in the disks that were NaOH passivated. Similar trends were also observed for the 99.9% Mg disks.

### Example 4

### Contact Angle

Contact angle tests and measurements were conducted on the disk samples, which were coated as described in Examples 1 and 2, to assess the hydrophobicity of the hybrid organosilane coating and the feasibility of timing the coating hydrophobicity by surface modification with 3-aminopropyl-trimethoxysilane (APES). The results are shown in Table 2.

TABLE 2
Contact Angle

| | Contact | | Contact angle |
|---|---|---|---|
| Treatment | angle (SD) | Treatment | (SD) |
| Mg bare | 59° (12) | AZ31 bare | 59 (2) |
| Mg NaOH passivated | 22° (2) | AZ31 coated | 109 (3) |
| Mg NaOH passivated coated | 111° (1) | AZ31 coated | 68.65 (9) |
| | | aminated | |
| Mg NaOH passivated coated | 67° (22) | | |
| aminated | | | |

The above results demonstrate that the hybrid organosilane coating significantly increased the hydrophobicity of the disks. Chemical surface modifications with APES can be used to regulate, control the hydrophobicity. High, hydrophobicity is a desirable property in cardiovascular applications, in particular, while hydrophilic surfaces are typically more suitable for bone applications. Other molecules including different functional groups e.g., thiols, carboxyls, phosphates, linked to a silane functionality also may be used for surface modification of the self-assembled organosilane films.

### Example 5

### SEM Characterization

SEM characterization was conducted to assess structural integrity of the hybrid organosilane films on the NaOH-treated Mg alloy surfaces. The SEM revealed a homogeneous smooth film covering an entire area of the disk. The film was scratched with a razor blade and analysis of the scratch revealed a laminar structure of the layer, which confirmed that the structural organization of the coating consisted of hydrophobic layers containing hydrocarbon aliphatic tails, interlaced with hydrophilic layers containing crosslinked silane heads. Further, the results of the SEM analysis confirmed that the self-assembled hybrid organosilane coatings were effectively produced on the surface of NaOH-treated Mg and that these coatings were smooth, uniform and formed a strong interface with the underlying substrate.

### Example 6

### ATR PTIR Analysis

ATR PTIR analysis was used to confirm the chemical composition of the coating layers. The spectra of the DTES/TMOS hybrid self-assembled coating showed profound alkyl peaks in 3000 cm⁻¹ region and Si—O absorption band at 1000 cm⁻¹. Following amination, a NH₃ absorption band appeared at ˜1500 cm⁻¹, which indicated the presence of amines on the surface of the coating. In general, the results of the ATR FTIR analysis confirmed the alkyl-silane chemical composition of the coating and demonstrated effective functionalization of the coating with APES based on the appearance of the strong amide peak.

### Example 7

### Protein Binding to the Coating Surface

Coating surface modifications with bioactive molecules, alkaline phosphatase (ALP), were conducted and evaluated. Six Mg disks were coated with the DTES/TMOS hybrid self-assembled film and three of them were aminated with APES. The disks were incubated with ALP to induce covalent binding to the surface, rinsed, and ALP activity was assayed. The results showed that the ALP activity was higher for the APES-treated disks.

### Example 8

### Tissue Culture

Tissue culture experiments were conducted to assess the biocompatibility of the hybrid organosilane coatings. Twelve Mg disks were coated with the DTES/TMOS hybrid self-assembled film and six of them were aminated with APES. The disks were cultured with MC3T 3 cells in DMEM medium for 7 and 15 days. After 15 days, numerous cells were observed on the disks treated with APES while much fewer cells were observed on the disks which were not treated with APES. This difference in the cell density observed was attributed to the differences in hydrophobicity of the coating surfaces between the two experimental groups of disks. These cell experiments indicated the coatings were nontoxic, biocompatible, and that the cell response to the coating may be regulated/controlled by surface modifications. It was contemplated that surface modifications allow coatings to be optimized for different applications. For example, hydrophobic coatings could be used for cardiovascular applications, while for other sites/applications, such as bone, the coatings may be more hydrophilic.

### Example 9

### Cell Culture Experiment

Twelve Mg disks were coated with the self-assembled organosilane film. Six of them were further functionalized with 3-aminopropyl-trimethoxysilane (APES). The twelve disks were seeded with MT3T3 cells and cultured in α-MEM tissue culture medium for 21 weeks. After 15 days in culture, it was observed that cells were present on both the animated and non-animated disks and they appeared healthy. The MC3T3 cells had normal morphology and well-developed actin cytoskeleton. These observations suggested that the disks were biocompatible and promoting cell adhesion and proliferation. The cell density on the organosilane coated animated disks was 28.40+/−0.73 cell/10,000 μm², which was significantly higher (p<0.01) than the cell density of 17.83+/−1.72 cell/10,000 μm² on the organosilane-only coated disks. These results suggested that by modifying the surface properties of the coatings, cell response could be modulated. Thus, in general, the results demonstrated biocompatibility of the coating of the invention and feasibility of surface functionalization as a mechanism for modulating cell response.

### Example 10

### Hydrogen Release Experiment

Mg and AZ31 disks were obtained and evaluated for hydrogen release. The disks were polished and etched. A control disk was left uncoated. Other disks were prepared as follows: (i) NaOH treated, (ii) NaOH treated and coated with alkylsilane (AS), and (iii) NaOH treated, coated with AS and functionalized with APES.

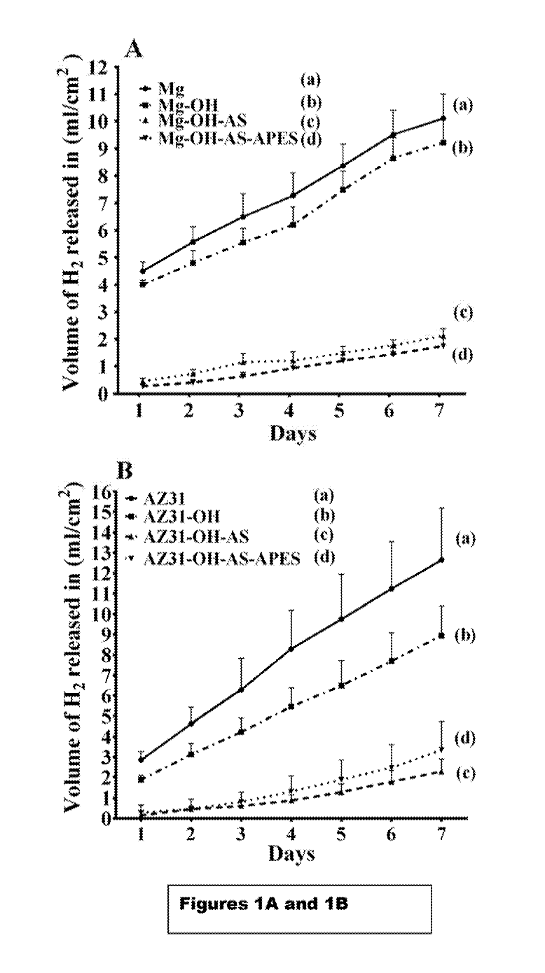

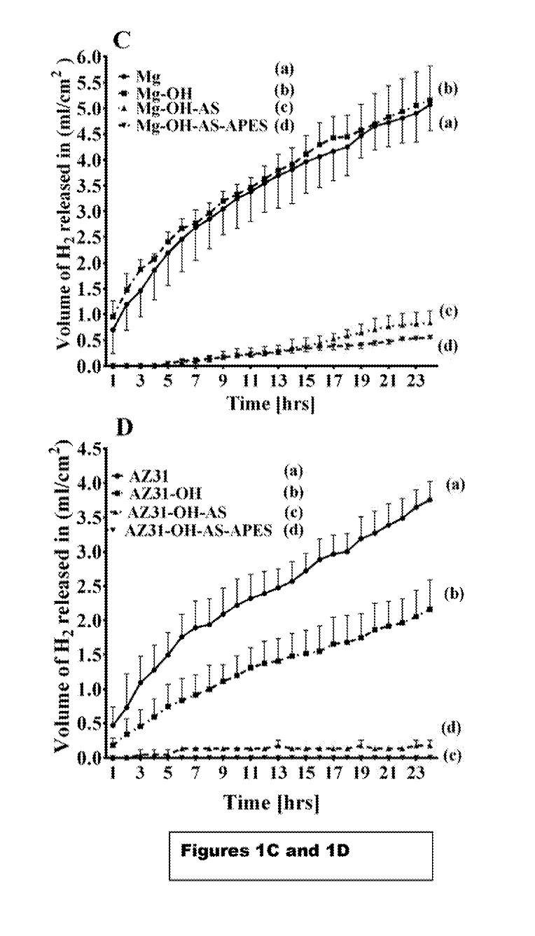

It was observed that cumulative H₂ released from the uncoated Mg disks over a period of 7 days was about 10 fold higher than Mg disks that were AS coated. FIGS. 1A, 1B, 1C and 1D show cumulative hydrogen release profiles from Mg and AZ31samples (shown in FIGS. 1A/1C and 1B/1D, respectively), following one week incubation (FIGS. 1A and 1B) in simulated body fluid (SBF) and 24 hours (FIGS. 1C and 1D). The difference was significant (p>0.0001). The alkali treatment slightly but significantly reduced the rate of if; release (p<0.0001) while the hydrogen release profile of the Mg—OH—AS and Mg—OH—AS-APES groups was not significantly different (p>0.0914). See FIG. 1A, Functionalized AS-coated Mg disks similarly showed significant reduction in hydrogen release compared to non-coated Mg disks. Experiments with AZ31 samples yielded similar results. The results are shown in Table 3 below.

To assess the ability of the AS coating to slow down the initial burst of corrosion, H₂ evolution studies were conducted over a 24-hour period after immersion of the disks in simulated body fluid (SBF). See FIG. 1C. The results of these experiments indicate that the AS coating reduced the initial Mg corrosion rate 5 fold from 0.16 ml/h to 0.03 ml/h, effectively preventing the initial burst of corrosion. After the initial 24 hours, the corrosion rate of both coated and non-coated samples significantly decreased and achieved steady state. Nevertheless, the corrosion rate of the AS-coated Mg was 4 times Sower than that of the bare Mg, 0.01 ml/h vs. 0.04 ml/h. The results of the experiments with AZ31 were very similar to those obtained with Mg (see FIG. 1B and 1D). These results clearly demonstrate the effectiveness of the AS coating to prevent the initial corrosion burst as well as to reduce the overall corrosion rate.

TABLE 3
Cumulative hydrogen evolution from the Mg and AZ31 samples.

| | | Cumulative hydrogen evolution rate (ml/hr) (mean ± SD) | | | |
|---|---|---|---|---|---|
| Sr. No | Time | Mg | Mg—OH-AS | AZ31 | AZ31-OH-AS |
| 1) | Day 1 (24 h) | 0.159 ± 0.014 | 0.026 ± 0.006 | 0.118 ± 0.007 | 0.005 ± 0.002 |
| 2) | Days 2-6 (144 h) | 0.039 ± 0.005 | 0.011 ± 0.001 | 0.068 ± 0.013 | 0.014 ± 0.003 |

### Example 11

### Weight Loss Experiments

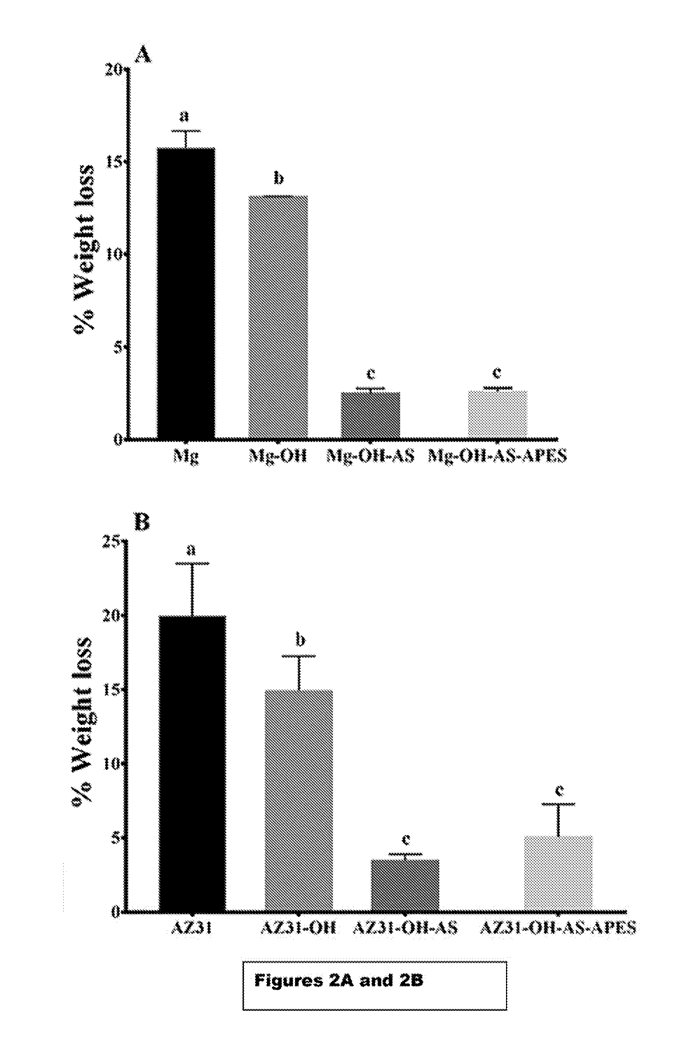

The results of the weight loss measurements corresponded to the hydrogen release experiments. FIGS. 2A and 2B show the percent of weight loss of Mg and AZ31, samples (shown in FIGS. 2A and 2B, respectively) after a 7-day incubation in SBF. The letter designations in FIGS. 2A and 2B indicate statistical significance (p<0.05). Columns marked with the same letters are not statistically different, while the columns marked with different letters are statistically different. Both Mg and Mg—OH samples showed significantly higher % weight loss compared to Mg—OH—AS and Mg—OH—AS-APES samples (see FIG. 2A). The weight loss of the Mg—OH was slightly but significantly smaller than the Mg group. Weight loss measurements of AZ31 samples also yielded similar results (see FIG. 2B).

### Example 12

### in vivo Experiments in the Subcutaneous Mouse Model

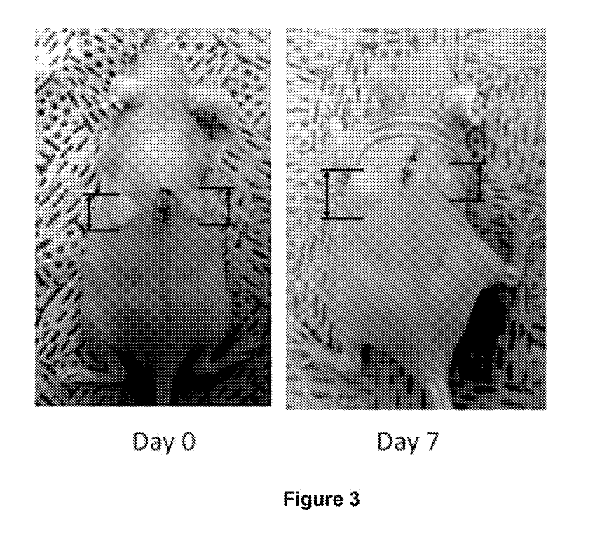

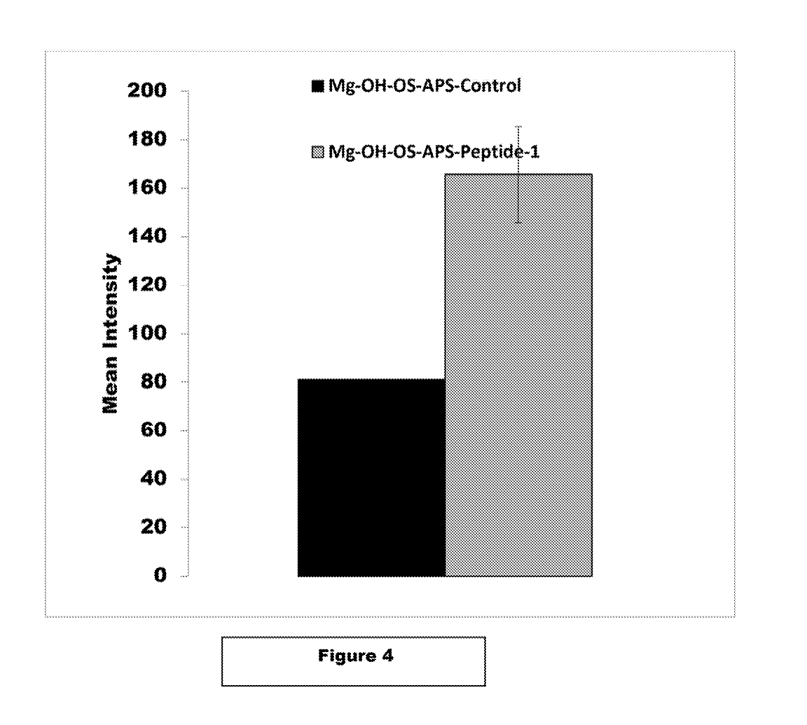

Five 4-week old nude mice were implanted subcutaneously with two (3-mm in diameter) Mg disks. Bare Mg disks were implanted into the left dorsal side and Mg—OH—AS disks were implanted into the right dorsal side. The diameters of the elevated skin areas covering the disks were measured on the day of operation and on Day 7 post-operation. While the diameters of the elevated skin areas around the coated disks remained unchanged, gas pockets formed around the uncoated disks (see FIG. 3). The measurements revealed that the diameters of the elevated areas around the hare Mg increased by 61% on average while the diameters of the elevated areas around the Mg—OH—AS samples remained virtually unchanged. The differences were highly statistically significant (p=0.0002). Overall, these results demonstrated that the AS coating is capable of effectively preventing the formation of gas pockets around implantable Mg devices.

### Example 13

### Fluorescence Intensity

Fluorescently labeled peptide was successfully covalently linked to the Mg—OH—AS surface via APES and triethoxysilane-PEG-maleamide linkers. Fluorescence intensity measurements were obtained from the AS-coated Mg samples functionalized with APES and triethoxysilane-PEG-maleamide and incubated with the fluorescent peptide. The results shown in FIG. 4 demonstrate that fluorescence intensity of the AS-coated-APES-functionalized samples incubated with the fluorescent peptide was significantly higher than the control sample, which suggested that the peptide was bound to the coating surface.

1. A medical implant device, comprising:
substrate comprising metal and having an outer surface;
self-assembled organosilane-containing coating applied to the substrate;
binding compound combined with the coating; and
active component coupled to the binding compound.

2. The device of claim 1, wherein the metal is selected from the group consisting of magnesium and magnesium alloy.

3. The device of claim 1, further comprising pretreatment applied to the outer surface of the substrate and the coating being applied to the pretreatment.

4. The device of claim 1, wherein the coating comprises alkyltriethoxysilane.

5. The device of claim 4, wherein the alkyltriethoxysilane has a tail comprising C₄-C₂₀ aliphatic backbone and a silane head.

6. The device of claim 1, wherein the coating comprises a co-polymer of decyltriethoxysilane and tetramethoxysilane.

7. The device of claim 1, wherein the binding compound comprises 3-aminopropyl-trimethoxysilane.

8. The device of claim 3, wherein the pretreatment is selected from the group consisting of polishing with nitric acid, etching with nitric acid, passivating with sodium hydroxide, and combinations thereof.

9. The device of claim 1, wherein the binding compound is selected from the group consisting of amine, carboxyl, thiol, hydroxyl and mixtures thereof.

10. The device of claim 1, wherein the binding compound is coupled to a surface of the coating.

11. The device of claim 1, wherein the binding compound is mixed with the composition that forms the coating.

12. A method of preparing a medical implant device, comprising;
obtaining a uncoated substrate having au outer surface,
preparing a coating composition, comprising organosilane;
applying the coating composition to the uncoated substrate to form a coating thereon;
functionalizing the coating with a binding compound; and
coupling an active component to the binding compound.

13. The method of claim 12, wherein the applying of the coating composition is conducted by a deep-coating process at ambient temperature.

14. The method of claim 13, wherein the preparing and applying comprises:
combining organosilane and solvent to form a solution;
applying the solution to the uncoated substrate by dipping or immersing the substrate into a bath of the solution for a time sufficient for the organosilane to bond to the substrate;
evaporating the solvent;
inducing the organosilane to self-assemble into a micro- or nano-structure; and
forming a thin film coated substrate.

15. The method of claim 12, further comprising pretreating the surface of the uncoated substrate prior to the applying of the coating composition.
